(19) United States
(12) Patent Application Publication
(10) Pub. No.: US 2025/0332326 A1
(43) Pub. Date: Oct. 30, 2025

(54) METHOD OF GENERATING 3D POROUS HYBRID PROTEIN NANOSCAFFOLD

(71) Applicants: Rutgers, The State University of New Jersey, New Brunswick, NJ (US); Sung Kwang Medical Foundation, Seoul-si (KR)

(72) Inventors: Ki-Bum Lee, Monmouth Junction, NJ (US); Letao Yang, Piscataway, NJ (US); Inbo Han, Jeju-do (KR)

(73) Assignees: Rutgers, The State University of New Jersey, New Brunswick, NJ (US); Sung Kwang Medical Foundation, Seoul-si (KR)

(21) Appl. No.: 19/192,702

(22) Filed: Apr. 29, 2025

Related U.S. Application Data
(60) Provisional application No. 63/640,514, filed on Apr. 30, 2024.

(51) Int. Cl. A61L 27/54; A61L 27/02; A61L 27/26; A61L 27/56; A61L 27/58

(52) U.S. Cl. CPC A61L 27/54; A61L 27/025; A61L 27/26; A61L 27/56; A61L 27/58; A61L 2300/434; A61L 2400/12; A61L 2430/38

(57) ABSTRACT

Provided are biodegradable 3D porous hybrid protein (3D-PHP) nanoscaffolds that comprise MnO₂ nanosheets and avoid covalent modification of proteins. These nanoscaffolds demonstrate inflammatory stimuli-responsive drug release as well as disc-mimetic stiffness. Also provided are methods of using (e.g., treating intervertebral disc disease (IVDD)) and manufacturing such nanoscaffolds.

### CROSS-REFERENCE TO RELATED APPLICATIONS

This application is entitled to priority pursuant to 35 U.S.C. § 119 (e) to U.S. Provisional Application No. 63/640,514, filed on Apr. 30, 2024. The content of the application is incorporated herein by reference in its entirety.

### STATEMENT REGARDING FEDERALLY SPONSORED RESEARCH

This invention was made with government support under grant number CHE-1429062 awarded by the National Science Foundation (NSF). The government has certain rights in this invention.

### FIELD OF THE INVENTION

This invention relates to self-therapeutic 3D porous hybrid protein (3D-PHP) nanoscaffolds for the delivery of therapeutic agents as well as methods of manufacturing such nanoscaffolds.

### BACKGROUND OF THE INVENTION

Oxidative stress can lead to dysregulated immune cell responses that further result in chronic inflammation, which is a condition that has been linked to various severe medical conditions, including cardiovascular, neurological, and musculoskeletal diseases. Nevertheless, there is no efficient treatment or a clear understanding of these conditions at this time. Typically, inflammatory signaling in these diseases or injuries is complicated by various factors, including altered lipid metabolism, cell-free nucleic acids (cf-NAs) production, and increased levels of reactive oxygen species (ROS); additionally, epigenetic changes in the inflammatory genome are also key contributors to this complexity. However, effectively and dynamically modulating these complex microenvironmental and epigenetic elements remains a highly challenging task. For instance, the prevalence of intervertebral disc disease (IVDD), a common disorder in the United States and around the world, is rapidly increasing with the rapid aging of our society. Severe IVDD is often associated with chronic inflammation, a key source of back pain. Unfortunately, there is no widely adaptable strategy yet for effectively reducing unfavorable inflammatory signals in IVDD. Treating severe IVDD is complicated by inflammation-induced tissue fibrosis, extracellular matrix (ECM) degradation, and dysregulated immunometabolism. Thus, developing effective strategies to address these issues and restore proper inflammatory signaling is a critical area of research.

Current therapeutic options for late-stage IVDD focus primarily on alleviating symptoms, such as pain, without addressing all critical mediators of chronic inflammation. Non-steroidal anti-inflammatory drugs (NSAIDs) are often used for this purpose, but do not effectively suppress all critical inflammatory pathways. Regardless of the specific types of mediators investigated, anti-inflammatory drugs targeting epigenetics have been shown to have greater potential. However, their potential adverse effects on disc cell development limit their applicability to IVDD. Stem cell treatments, including stem cell-derived exosomes, have also been explored to partially restore a healthy microenvironment in IVDD. These treatments target inflammatory signaling pathways and replenish disc cell populations in the affected area; nonetheless, their efficacy is highly dependent on stem cell survival and differentiation or the preservation of exosome populations. This necessitates more creative and effective ways of adjusting immune cells in IVDD. Given these challenges, there is a need to develop a multimodal and innovative therapeutic approach to improve the treatment of IVDD and other inflammation-associated tissue injuries and diseases. Such an approach would address the disease's multifaceted nature, allowing immune cells to be effectively regulated in a safer and more sustainable manner.

Multi-functional and multi-dimensional biomaterials, particularly self-therapeutic for scavenging inflammatory mediators, generating pro-regenerative ECM, or protecting tissue from apoptotic signals, are regarded as potentially valuable platforms for long-term immunomodulation and efficient tissue regeneration. To date, achieving sustainable, dynamic, and efficient anti-inflammation for ECM and cellular microenvironment restoration in IVDD has proven difficult.

### SUMMARY OF THE INVENTION

The present disclosure relates to biodegradable 3D porous hybrid protein (3D-PHP) nanoscaffolds that comprise MnO₂ nanosheets and avoid covalent modification of proteins. Also provided are methods of using (e.g., treating intervertebral disc disease (IVDD)) and manufacturing such nanoscaffolds.

In one aspect, provided is a 3D porous hybrid protein (3D-PHP) nanoscaffold comprising biodegradable manganese dioxide (MnO₂) nanosheets assembled with aqueous cationic polymer solutions and extracellular matrix proteins, wherein the nanoscaffold comprises at least 100 layers, wherein the size of the cationic polymers is greater than 100 kDa, wherein the concentration of the cationic polymers is greater than 1% (weight percent to water), and wherein the nanoscaffold further comprises a therapeutic agent. In some embodiments, the cationic polymers comprise chitosan, polyphenylalanine, polytryptophan, polyasparagine, polyglutamine, polylysine, polyarginine, polyhistidine, polyethylenimine, or poly (amidoamine). In one embodiment, the nanosheets are assembled into the 3D-PHP nanoscaffold via an electrostatically driven layer-by-layer (LBL) 3D assembly technique and wherein the ratio of MnO₂ to proteins is about 9:1. In one embodiment, the cationic polymer solution concentration is about 20% to about 70%, the concentration of MnO₂ is about 20% to about 70%, and the concentration of the proteins is about 5% to about 15%.

In some embodiments, the extracellular matrix proteins comprise collagen I, collagen II, laminin, and fibronectin.

In one embodiment, the viscosity of the aqueous cationic polymer solution is greater than 20 cP (centipoise).

In one embodiment, the pore sizes range from about 5 μm to about 50 μm.

In one embodiment, the nanoscaffold further comprises a bromodomain extraterminal inhibitor (BETi). In one embodiment, the nanoscaffold comprises at least 50 μg/mL BETi.

In one embodiment, the nanoscaffold delivers the therapeutic agent to a subject in need thereof. In some embodiments, the therapeutic agent is selected from the group consisting of small molecules, biologics, nucleic acids, and cells. In one embodiment, the cells are stem cells.

In one aspect, provided is a method of treating intervertebral disc (IVD) degeneration in a subject in need thereof, the method comprising administering to said subject a 3D porous hybrid protein (3D-PHP) nanoscaffold comprising biodegradable manganese dioxide (MnO₂) nanosheets assembled with aqueous cationic polymer solutions and extracellular matrix proteins, wherein the nanoscaffold comprises at least 100 layers, wherein the size of the cationic polymers is greater than 100 kDa, wherein the concentration of the cationic polymers is greater than 1% (weight percent to water), and wherein the nanoscaffold further comprises a therapeutic agent.

In another aspect, provided is a method of treating a chronic inflammation-related disease or condition in a subject in need thereof, the method comprising administering to a subject in need thereof a 3D porous hybrid protein (3D-PHP) nanoscaffold comprising biodegradable manganese dioxide (MnO₂) nanosheets assembled with cationic polymers and extracellular matrix proteins, wherein the nanoscaffold comprises at least 100 layers, wherein the size of the cationic polymers is greater than 100 kDa, and wherein the concentration of the cationic polymers is greater than 1% (weight percent to water). In some embodiments, the chronic inflammation-related disease or condition comprises osteoarthritis, rheumatoid arthritis (RA), neuroinflammation, Alzheimer's disease, spinal cord injury, sepsis, stroke, gout, psoriatic arthritis, myositis, scleroderma, an autoimmune disease producing inflammatory levels of reactive oxygen species (ROS), and cancer.

### BRIEF DESCRIPTION OF THE DRAWINGS

FIGS. 1A, 1B, 1C, 1D, 1E, and 1F show the generation and modulation of 3D-PHP nanoscaffolds for drug delivery and intervertebral disc tissue engineering. FIG. 1A shows scanning electron microscopy (SEM) images comparing the morphological and structural changes of chitosan nanoscaffold, chitosan-MnO₂ nanoscaffold, and 3D-PHP nanoscaffold. FIG. 1B shows the tuning of the pore sizes and porosity of the 3D-PHP nanoscaffolds by varying the concentrations of initial nanosheets used for the assembly from 1.8 mg/mL, 2.4 mg/mL, to 3.0 mg/mL. FIG. 1C illustrates atomic force microscopy (AFM)-based measurements of stiffness of 3D-PHP nanoscaffold with medium pore size that showed similar range of mechanical property as the nucleus pulposus region in the intervertebral disc tissues. FIG. 1D is LC-MS characterization of the JQ1 molecule, which shows a single distinctive peak at 3.48 minutes with molecular weight of 457 g/mol. The inset image shows the structure of JQ1. FIG. 1E shows the standard curve of JQ1 in the LC-MS characterization of drug concentration. The high linearity and R-square of 0.996 justifies its use for quantification of JQ1 release. FIG. 1F shows pH-dependent release of JQ1 from 3D-PHP nanoscaffold. The graph indicates the faster release from a slight change of neutral to acidic pH.

FIGS. 2A and 2B show transmission electron microscopy (TEM) characterization of MnO₂ nanosheets. FIG. 2A shows schematic diagrams of the top and side view of the nanosheet showing the birnessite crystal structure. FIG. 2B is a graph summarizing the size distribution of nanosheets.

FIG. 3 shows X-ray photoelectron spectroscopy (XPS) characterization of the nanosheet and nanoscaffolds. The presence of manganese peak confirms the successful incorporation of nanosheet within the nanoscaffold during the assembly process.

FIG. 4 shows zeta potential-based confirmation of successful functionalization of nanosheet with gelatin and chitosan during the assembly into nanoscaffolds. Nanosheet with a negative charge become neutrally charged after loading of gelatin, and further shifted to positively charged after assembly with chitosan, which is a common biocompatible cationic polymer.

FIGS. 5A and 5B shows the pH, structure- and composition-dependent drug release from the nanoscaffolds. FIG. 5A depicts drug release that was performed in phosphate-buffered saline (PBS); FIG. 5B depicts drug release that was performed in pH 6.5 buffer. Square: gelatin-incorporated 3D-PHP scaffold with high porosity; dot: gelatin-incorporated 3D-PHP scaffold with medium porosity; triangle (up): gelatin-incorporated 3D-PHP scaffold with low porosity; triangle (down): MnO₂ nanoscaffold with high porosity. This endows further tunability of drug release and inflammation modulation to the scaffold system.

FIGS. 6A, 6B, 6C, 6D, 6E, 6F, 6G, and 6H depict dual scavenging of ROS and cf-NAs by 3D-PHP nanoscaffolds. FIG. 6A is a schematic diagram showing the diverse inflammatory mediators, including ROS and cf-NAs after an initial injury to the intervertebral disc tissues that cause massive cell apoptosis. FIG. 6B shows the mechanism of MnO₂ nanosheet-mediated scavenging of ROS and reduction of inflammation. FIG. 6C shows quantification of H₂O₂ scavenging efficiency of different MnO₂ nanomaterials. FIG. 6D is schematic diagram showing the electrostatic binding between cationic chitosan polymers and anionic cf-NAs. FIG. 6E depicts a YOYO1-based assay for characterizing the binding of cf-NAs (CpG) by 3D-PHP nanoscaffold and control scaffolds. FIGS. 6F, 6G, and 6H show qRT-PCR analysis of inflammatory genes (TNF and IL8) after treatment by PBS (control condition 1, FIG. 6F), chitosan only (control condition 2, FIG. 6G), and 3D-PHP nanoscaffold (experimental condition, FIG. 6H). N=3 biological replicates, *P<0.05.

FIG. 7 depicts qRT-PCR analysis of the survival and apoptosis related genes of disc cells (annulus fibrosus cells and nucleus pulposus cells) after incubation with condition media collected from macrophages treated with LPS, free BETi solution, 3D-PHP nanoscaffolds loaded with different amounts of BETi.

FIGS. 8A, 8B, 8C, 8D, 8E, 8F, and 8G show 3D-PHP nanoscaffold-based delivery of BETi for anti-inflammation in macrophages. FIGS. 8A, 8B, 8C, 8D, 8E, and 8F show qRT-PCR analysis of inflammatory (TNF (FIG. 8A), IL6 (FIG. 8B), IL8 (FIG. 8C), CXCL1 (FIG. 8D), CCL5 (FIG. 8E) and anti-inflammatory (IL4; FIG. 8F) gene expression after treatment of human THP1 monocyte-derived macrophages by PBS (ctrl), LPS, 3D-PHP nanoscaffold loaded with low, medium, and high concentrations of BETi. FIG. 8G shows the reduction of apoptotic cells in disc cell culture after incubation with condition media extracted from LPS-stimulated macrophages treated by 3D-PHP nanoscaffold loaded with different amount of BETi. The white/black image is pseudo colored from dead cell staining. N=3 biological replicates, *P<0.05.

FIG. 9 shows the anti-inflammation outcome of 3D-PHP nanoscaffold in a rat nucleotomy disc injury model. FIG. 9 depicts graphs of the quantification of histological analysis on healthy disc tissue area (based on H&E staining), IL-1β (from immunostaining, detailed images are in the supplementary information), MMP (from MMP13 immunostaining). These graphs support 3D-PHP nanoscaffold-based strategy for suppressing inflammation in vivo in a rat nucleotomy disc injury model. Each dot indicates a biological replicate, *P<0.05.

FIG. 10 shows the MRI analysis of histological outcome of 3D-PHP nanoscaffold treatment and the quantification of MRI index.

FIG. 11 depicts in vivo characterization and quantification of inflammatory marker (percentage of TNF positive cells as compared to DAPI positive cells).

FIG. 12 shows anti-inflammation mediated intervertebral disc tissue regeneration through ECM remodeling and disc cell protection. FIG. 12 shows graphs of the quantification of histological analysis on ECM restoration (based on the percentage of Collagen II positive area) and disc cell protection or regeneration (based on percentage of Brachyury and Tie2 positive cells). Each dot indicates a biological replicate, *P<0.05.

FIG. 13 shows the histological analysis of aggrecan expression 6WPI (weeks post-injury) of implantation of 3D-PHP nanoscaffolds and control conditions.

FIGS. 14A and 14B show that 3D-PHP nanoscaffold reduces pain associated with rat disc degeneration. FIG. 14A is a summary of Von-Frey tests after rats treated by the different indicated conditions. FIG. 14B shows quantification on the histological analysis of pain related marker (CGRP) positive cell percentage. Each dot indicates a biological replicate, *P<0.05.

FIG. 15 depicts in vivo characterization and quantification of nerve growth factor (NGF) positive cells that are associated with the generation of discogenic pain and pain reduction by 3D-PHP nanoscaffolds.

### DETAILED DESCRIPTION OF THE INVENTION

### 1. Overview

The present disclosure relates to biodegradable 3D porous hybrid protein (3D-PHP) nanoscaffolds that comprise MnO₂ nanosheets for delivering therapeutic agents to a subject in need thereof. In some embodiments, the therapeutic agents include, but are not limited to small molecules, biologics, nucleic acids, and cells. Also provided are methods of manufacturing the nanoscaffolds described herein. In some embodiments, the nanoscaffolds are used to treat a chronic inflammation-related disease or condition in a subject in need thereof. In one embodiment, the nanoscaffolds described herein are used to treat intervertebral disc (IVD) degeneration in a subject in need thereof.

In some embodiments, the nanoscaffolds provide long-lasting scavenging of reactive oxygen species (ROS) and cell-free nucleic acid (cf-NA) for effective anti-inflammatory therapy. Degeneration of fibrocartilaginous tissues is often associated with complex pro-inflammatory factors that includes ROS, cf-NAs, and epigenetic changes in immune cells. Thus, the presently described nanoscaffolds can control these inflammatory signals.

As used herein, the term “nanosheets” refers to a sheet of material that has a thickness of about 1 to about 100 nanometers and is two-dimensional (2D).

As used herein, the term “nanoscaffolds” refers to three-dimensional (3D) structures composed of nanoscale features.

As used herein, the term “subject” refers to an animal, preferably a mammal such as a human. The terms “subject” and “patient” can be used interchangeably.

As used herein, the term “small molecule” may refer to non-peptidic, non-oligomeric organic compounds, either synthesized or found in nature. These compounds may be “natural product-like,” however, the term “small molecule” is not limited to “natural product-like” compounds. Small molecules are typically characterized in that they possess one or more of the following characteristics: several carbon-carbon bonds, multiple stereocenters, multiple functional groups, at least two different types of functional groups, and a molecular weight of less than 1500, although not all, or even multiple, of these features need to be present.

As used herein, the term “biologics” can refer to growth factors, immune modulators, vaccines, antibodies, and products derived from human blood and plasma. Biologics can be produced from living organisms or contain components of living organisms.

As used herein, the term “nucleic acid,” may refer to a polymer composed of a multiplicity of nucleotide units (ribonucleotide, deoxyribonucleotide, or related structural variants) linked via phosphodiester bonds, including but not limited to, DNA or RNA. The term encompasses sequences that include any of the known base analogs of DNA and RNA. Examples of a nucleic acid include, and are not limited to, mRNA, miRNA, tRNA, rRNA, snRNA, siRNA, dsRNA, cDNA and DNA/RNA hybrids. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil (U), adenine (A), thymine (T), cytosine (C), guanine (G), and their derivative compounds. Nucleic acids may be obtained by chemical synthesis methods or by recombinant methods. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

As used herein, the term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. The terms “prevent” or “preventing” refer to prophylactic and/or preventative measures, wherein the object is to prevent, or slow down the targeted pathologic condition or disorder. In addition, “treating” or “treatment” does not require complete alleviation of signs or symptoms, does not require a cure, and specifically includes protocols that have only a marginal effect on the patient.

As used herein, and in the appended claims, the singular forms “a”, “and” and “the” include plural references, unless the context clearly dictates otherwise.

The term “about” refers to a range of values which would not be considered by a person of ordinary skill in the art as substantially different from the baseline values. For example, the term “about” may refer to a value that is within 20%, 15%, 10%, 9%, 8%, 7%, 6%, 5%, 4%, 3%, 2%, 1%, 0.5%, 0.1%, 0.05%, or 0.01% of the stated value, as well as values intervening such stated values. Context will dictate which value, or range of values, the term “about” may refer to in any given instance, throughout this disclosure.

Where a value of ranges is provided, it is understood that each intervening value, between the upper and lower limit of that range, and any other stated or intervening value in that stated range, is encompassed within the invention. The upper and lower limits of these smaller ranges, which may independently be included in the smaller ranges, is also encompassed within the invention, subject to any specifically excluded limit in the stated range.

All publications mentioned herein are incorporated herein by reference in their entireties.

Unless otherwise defined, all technical and scientific terms used herein have the same meaning as commonly understood by one of ordinary skill in the art to which this invention pertains. In the case of conflict, the present document, including definitions will be controlled.

### 2. 3D Porous Hybrid Protein (3D-PHP) Nanoscaffolds

In some embodiments, the 3D-PHP nanoscaffold described herein comprise biodegradable manganese dioxide (MnO₂) nanosheets assembled with aqueous cationic polymer solutions, wherein the 3D-PHP nanoscaffold avoids covalent modification of proteins. Examples of cationic polymers include, but are not limited to chitosan, polyphenylalanine, polytryptophan, polyasparagine, polyglutamine, polylysine, polyarginine, polyhistidine, polyethylenimine, or poly (amidoamine). In some embodiments, the ratio of MnO₂ to proteins is about 9:1.

In some embodiments, the nanoscaffold described herein is self-therapeutic. As used herein, the term “self-therapeutic” refers to the fact that the nanoscaffold itself confers therapeutic properties to a subject such as reducing reactive oxygen species, removing cell-free nucleic acids, and reducing inflammation.

In some embodiments, the pore sizes of the nanoscaffold are modulated by the concentration of the nanosheets. In some embodiments, the pore sizes range from about 5 μm to about 50 μm.

In one embodiment, the viscosity of the aqueous cationic polymer solution is greater than 20 cP (centipoise). Viscosity can be measured using any method known in the art such as the American Society for Testing and Materials (ASTM) 2196-20 standard (astm.org/d2196-20.html). In particular, Test Method A is used to determine the apparent viscosity at a given rotational speed. In the case of Test Methods B and C, the extent of shear thinning is indicated by the drop in viscosity with increasing rotational speed. Test Methods A-B all involve the determination of the apparent viscosity and the shear thinning and thixotropic properties of non-Newtonian materials in the shear rate range from 0.1 s⁻¹ to 50 s⁻¹ using a rotational viscometer operating in a fluid contained in a 600 mL low form Griffin beaker. The function between force and distance is used to calculate the viscosity of the polymer solution.

In one embodiment, the cationic polymer solution concentration is about 20% to about 70%, the concentration of MnO₂ is about 20% to about 70%, and the concentration of the proteins is about 5% to about 15%.

In some embodiments, the biodegradable nanoscaffold described herein may include one or more therapeutic agents. In this embodiment, one or more of the therapeutic agents may be trapped, or embedded in the nanoscaffold. The therapeutic agents may bind to, or associate with the nanoscaffold. This association may or may not be through interactions similar to that of ECM proteins with the manganese dioxide in the nanoscaffolding. The therapeutic agents may include, but are not limited to, any therapeutic agents that contain amine and/or aromatic functional groups/side chains. Such compositions are known to one of ordinary skill in the art. For example, therapeutic agents may include, but are not limited to, any of peptides, proteins, antibodies, nucleic acids, biologic drugs, small molecules, cells, cytokines, ligands, and combinations thereof. Other therapeutic agents include, purely by way of example, chemotherapeutic agents, antipyretics, analgesics/anesthetics, antibiotics, antiseptics, hormones, stimulants, depressants, statins, beta blockers, anticoagulants, antivirals, anti-fungals, anti-inflammatoirewth factors, vaccines, diagnostic compositions, psychiatric medications/psychoactive compounds, and any related compositions. In some embodiments, the nucleic acids include, but are not limited to siRNA or antisense DNA.

In one embodiment, cells may be disposed in and on the nanoscaffold. In one embodiment, these cells may be stem cells, such as for example, embryonic stem (ES) cells, adult stem cells, induced pluripotent stem (iPS) cells, induced somatic stem cells (iSC) and combinations thereof. More specifically, the stem cells can include hematopoietic stem cells (HSCs), mammary stem cells, intestinal stem cells, mesenchymal stem cells (MSCs), endothelial stem cells, neural stem cells (NSC), olfactory adult stem cells, neural crest stem cells, testicular cells, adipose-derived stem cells (ADSCs), and combinations thereof. In an exemplary embodiment, the stem cells may be neural stem cells (NSCs), e.g., for treatment of traumatic brain injury. The stem cells may undergo differentiation while embedded in the scaffolding material. This process may be, but is not necessarily, directed by the presence of specific extracellular matrix (ECM) proteins. For example, nanoscaffolds containing laminin may promote differentiation of neural cells, which are useful for treatment of spinal cord injury (SCI), as illustrated by the Examples. Those containing fibronectin may promote myogenesis (differentiation of muscle cells) and osteogenesis (differentiation of bone cells). Meanwhile, nanoscaffolds containing aginate may promote neurogenesis (differentiation of neural cells). One of skill in the art will recognize that there are a number of ECM proteins, including but not limited to those disclosed herein, which may result in different stem cell differentiation. The nanoscaffolds may thus be used for autologous grafting, e.g., autologous nerve grafting, allografting, or even xenografting.

In some embodiments, the nanoscaffold further comprises a bromodomain extraterminal inhibitor (BETi). In some embodiments, the nanoscaffold comprises at least 50 g/mL BETi. In some embodiments, the nanoscaffold comprises about 50 μg/mL BETi to about 200 g/mL BETi such as about 50 μg/mL BETi, about 100 μg/mL BETi, about 150 μg/mL BETi, or about 200 μg/mL BETi.

The biodegradable MnO₂ nanosheet that comprises the nanoscaffold described herein is crucial for tissue engineering, and the degradation product (Mn(II)) provides magnetic resonance imaging (MRI) enhancement. MnO₂ nanosheets degrade in the presence of cell metabolism outputs, such as ascorbic acid, according to a classic reduction-oxidation mechanism. In vivo, the main mechanism for controlling the rate of degradation of the MnO₂ nanosheets is the porosity of the scaffold. The rate of degradation of the MnO₂ nanosheets may also be controlled by other means, such as for example, controlling the thickness of the MnO₂ layers in the nanoscaffold, the aspect ratio (height to surface area ratio) of the nanoscaffold, the extracellular matrix protein concentration, the concentration of reductants, modifying interlayer binding species (for example, ions and proteins, e.g., but not limited to spacer proteins, including bovine serum albumin) or the cellular density.

In one embodiment, the rate of biodegradation of the nanoscaffolds is tunable by changing the porosity of the scaffolding material. Likewise, the rate at which the therapeutic agent is released is also tunable. This is due to the fact that, the rate at which the therapeutic agent is released from the biodegradable nanoscaffolding is typically substantially equivalent to the rate at which the biodegradable scaffolding material is degraded in vivo.

The rate at which the biodegradable MnO₂-containing nanoscaffolding material is degraded in vivo can be measured by detecting the release of Mn⁺² ions from the biodegradable scaffolding material (for example by MRI or FRET). The nanoscaffolds release Mn⁺² on degradation, producing an MRI-detectable signal which can be used to quantify the degradation rate. As noted above, the rate at which the therapeutic agent or cells are released from the biodegradable nanoscaffolding, is typically substantially equivalent to the rate at which the biodegradable scaffolding material is degraded in vivo. Thus, the rate at which the therapeutic agent is released is measurable, by quantifying the rate/amount of Mn⁺² released. Additionally, because Mn⁺² is similar to Ca 2, it may be internalized by cells and retained, rather than being cleared immediately. Low dimension MnO₂ support structures also serve as fluorescent quenchers and enable detection of degradation and drug release with FRET.

### 3. Preparing 3D-PHP Nanoscaffolds

The nanoscaffold described herein is generated using a novel method for nanomaterial-templated protein assembly (NTPA). NTPA is a non-covalent method of synthesizing protein nanoscaffolds by immobilizing proteins to enzyme-like 2D MnO₂ nanosheets, which are co-assembled with cationic polymers into 3D nanoscaffolds. The MnO₂ nanosheets can be cleaved and degraded post-assembly. However, 3D-PHP nanoscaffolds can be used for in vivo transplantation without removing the MnO₂ nanosheets.

In summary, NTPA comprises the following steps:
1. non-covalent immobilization of ECM protein onto rigid MnO₂ biodegradable nanoscaffolds;
2. co-assembly with cationic polymer (e.g., chitosan) into porous nanoscaffolds by adding viscous droplet of the polymer solution with various concentrations of gelatin-bound nanosheets (or nanosheets bound with other ECM proteins);
3. co-assembly process can include small molecules, biologics, nucleic acids and/or cells; and
4. optionally, prior to translating the nanoscaffold, the MnO₂ nanosheet can be degraded.

### 4. Methods of Treatment or Use

The nanoscaffold of the present disclosure can be used to treat, or prevent a disease or disorder in a subject in need thereof. In one embodiment of the invention, the nanoscaffold can be surgically implanted, for example by grafting or inserting, into the subject. In a different embodiment, the nanoscaffold can be injected into the subject. Whether implanted or injected, the nanoscaffold would typically contain a therapeutic agent, such as those described herein-above. The diseases or disorders which the nanoscaffold of the present disclosure can be used to treat are explicitly not limited. The examples presented herein show treatment of intervertebral disc disease (IVDD), however, this is only one possible application.

In some embodiments, the nanoscaffold described herein can be used to a chronic inflammation-related disease or condition in a subject in need thereof. The chronic inflammation-related disease or condition includes, but is not limited to osteoarthritis, an autoimmune disease producing inflammatory levels of reactive oxygen species (ROS), rheumatoid arthritis (RA), and cancer. Examples of cancer include, but are not limited to anal cancer, bladder cancer, blood cancer, bone cancer, bone marrow cancer, colon cancer, breast cancer, cervical cancer, head and neck cancer, kidney cancer, lung cancer, liver cancer, ovarian cancer, pancreatic cancer, stomach cancer, skin cancer, prostate cancer, testicular cancer, and thyroid cancer.

In one embodiment, the nanoscaffold described herein can be used to treat traumatic brain injury (TBI).

In one embodiment, the nanoscaffold described herein is used to promote wound healing.

For example, the wound may have been sustained from a fall or cut. In one embodiment, the wound is infected. In some embodiments, the nanoscaffold described herein treats tissue injuries such as intervertebral disc, spinal cord, liver, bone, muscle, and brain injuries.

In one embodiment, the nanoscaffold described herein can scavenge reactive oxygen species (ROS) and cell-free nucleic acids (cf-NAs).

In one embodiment, the nanoscaffold described herein promotes restoration of the ECM. In some embodiments, administering the nanoscaffold described herein to a subject in need results in long-term pain reduction.

### EXAMPLES

### Example 1. Materials and Methods

This Examples details the materials and methods used in Examples 2-6.

### Example 1A. Synthesis of MnO₂ Nanosheets

2D manganese dioxide (MnO₂) nanomaterials were synthesized using a redox reaction between manganese acetate (II) salt and hydrogen peroxide based on a published protocol. Specifically, tetramethyl ammonium pentahydrate solution (abbreviated as TMAOH, 2.2 g in 10 mL ultrapure water) was added into 10 mL 6% hydrogen peroxide (H₂O₂). Manganese acetate solution (abbreviated as Mn(Ac)₂, 0594 g dissolved in 10 mL ultrapure water) was then added into TMAOH solution under vigorous stirring followed by slow stirring overnight. Dark-colored products immediately form in the solution and color increased during the progression of reaction. The initial step is often associated with rapid gas generation and can be dangerous, so it should be performed with caution. After the reaction was completed, the dark-colored precipitates were centrifuged down at 3000 rpm for 5 minutes and washed by water and ethanol for 3 times each followed by oven drying overnight. Birnessite MnO₂, which is the precursor of nanosheets, is formed at this stage. Afterward, a 10 mg/mL MnO₂ aqueous mixture was exposed to tip sonication (Brandson Sonics) at a 50% power output for 1 hour, then the solution was purified by centrifugation at 3000 rpm for 15 minutes and removal of the unsuspended precipitates. The concentration of the nanosheets was measured by drying 5.0 mL of solution in a vacuum oven followed by weighing the nanosheet weights. Hydrodynamic size and zeta potential of nanomaterials were studied by Malvern Instruments Nano Zetasizer series. Temperature was set at 25° C., and each value was averaged from at least 5 runs. Typical concentrations used in these measurements are 100 μg/mL.

### Example 1B. Loading Gelatin to Nanosheets

Gelatin was absorbed to nanosheets through non-covalent interactions such as electrostatic, hydrogen bonding, and metal-π interactions. Specifically, 10 mg/mL gelatin (from porcine skin, Type A, Sigma Aldrich) aqueous solution was prepared by dissolving 1.0 gram gelatin powder in 100 mL warm water. The solution was then added into 100 mL 3 mg/mL MnO₂ nanosheet solution in a dropwise manner followed by stirring overnight. Afterwards, 100 mL PBS was added into the solution followed by another 4 hours of stirring. Nanosheet loaded with gelatin was then centrifuged down at 10000 rpm for 15 minutes, and purified by washing with PBS and water 3 times each.

### Example 1C. Fabrication of 3D-PHP Nanoscaffolds and Control Scaffolds

To assemble nanosheets into 3D-PHP nanoscaffolds, an electrostatically driven layer-by-layer (LBL) 3D assembly technique reported by us and others was adapted. Specifically, a highly viscous droplet of chitosan solution with molecular weight of 30,000 to 50,000 g/mol and weight concentration of 2% was added into the bottom of a low-volume centrifuge tube used in PCR studies. Then 200 uL aqueous solutions with varying amount (1.2, 2.4, 3.0 mg/mL) of gelatin-coated nanosheets were added into the chitosan droplets. The cationic chitosan in the viscous droplet will slowly diffuse into the MnO₂ nanosheet solution and water trapped during the diffusion serves as templates for pore formation. After 12 hours of diffusion, the reaction was stopped and frozen at −80° C. followed by freeze drying overnight. For the control nanoscaffolds, including the nanoscaffold without gelatin, nanosheet was used directly without coating of gelatin. For chitosan scaffold, the chitosan droplet was mixed with 200 uL water followed by direct freeze drying. Nanoscaffolds were characterized by SEM for pore analysis. For the pore modulation the 3D-PHP nanoscaffold is self-assembled from anionic nanosheets and cationic polymer (i.e., chitosan) through electrostatically driven interfacial self-assembly previously reported by us and others. In this assembly mechanism, the concentration of MnO₂ nanosheet determines the diffusion speed and thus the size of water droplets encapsulated between different layers during diffusion. After lyophilization the pore would start to form and therefore the nanosheet concentration determines the pore sizes.

### Example 1D. Electron Microscopy and Photoluminescence Spectroscopy

Transmission electron microscopy (TEM) characterization on nanosheets was performed using a Philips CM12 TEM. Imaging was all performed at a voltage of 80 kilovolts (kv) and photos were taken with an AMT digital camera with the model number of XR111. Nanosheet samples were prepared by drop-casting 10 uL of 50 μg/mL nanosheet aqueous solution onto a holey carbon copper grid (EMS). Field-emission scanning electron microscopy (SEM) characterizations on nanoscaffolds and control scaffolds were performed using a Zeiss DSM 982 instrument with an electron voltage of 5 kV. Samples were placed on aluminum stubs and coated with 20 nm Au—Pd alloy using a sputter. X-ray photoluminescence spectroscopy (XPS) analysis on the nanoscaffold and nanosheet samples was performed using a Thermo Scientific ESCALAB 250 Xi model at a base pressure of 1E-9 or lower. For sample preparation, 100 μg/mL MnO₂ nanosheet aqueous solution was dried on a cleaned silicon substrate. Core level spectra was collected using a Monochromated X-ray source (Al-Ka) with an instrumental broadening of 0.5 eV.

### Example 1E. Atomic Force Microscopy-Based Stiffness Measurement of Nanoscaffold

The mechanical property (i.e., Young's modulus) of 3D-PHP nanoscaffold (assembled from 2.4 mg/mL nanosheet condition) was characterized by atomic force microscopy (AFM). A special hydrogel AFM tip was used for the stiffness measurement by a Park Systems NX-10 model. Using contact mode, scanning of nanoscaffold morphology was first performed followed by the force value measurement. Hydrogel AFM tips with fixed Young's modulus were used for the measurement. The Young's modulus was measured and calculated using standard equations integrated in the SmartScan software from Park Systems.

### Example 1F. Drug Loading and Release Study

Drug release profile of 3D-PHP nanoscaffolds were investigated using a model dye rhodamine B (RhB) for precise tracking. Specifically, equivalent amount of 100 μg RhB was added into the nanosheet solution before the assembly. Different conditions, including gelatin-coated and nanosheet-only, and nanosheet at varying concentrations, were then used for the formation of 3D-PHP nanoscaffolds and control nanoscaffolds with chitosan, as described in the section of nanoscaffold fabrication. After lyophilization, nanoscaffolds were briefly washed with PBS, then the drug release study was started at different buffer (pH 5, pH 6, and pH 7 phosphate buffer for the pH dependent study; the rest of the drug release studies performed in PBS). The drug released at each time point was collected and measured by UV-Vis in a plate reader (BioRad) using wavelength at 490 nm. New buffer was added at each time point after collection of the released drug solution.

### Example 1G. Monocyte Culture and Differentiation

THP-1 monocytes were purchased from ATCC® and cultured using standard protocol provided by the vendor (10% FBS in 1640 RPMI basal media supplemented with 50 nM beta-mercaptoethanol). Media was refreshed every other day. THP-1 monocytes were cultured at a density of 100,000 to 500,000 cells per mL. When they proliferate above the density, cells were centrifuged down for media change. To convert suspension culture of THP-1 monocytes into adherent macrophages, 200 ng/ml of phorbol myristate acetate (PMA) was added into the media then split into 24 well plates at designated density (100,000 per well). PMA was treated for 1-3 days then fresh growth media without PMA was added. To stimulate the macrophages, 1,000 ng/mL lipopolysaccharide (LPS, Sigma Aldrich) was included in the media for 4 hours.

### Example 1H. Disc Cell Culture

For the culture of disc cells, including nucleus pulposus (NP) cells and annulus fibrosus (AF) cells, cell culture plates were first coated with poly-l-lysine (PLL) aqueous solution at a concentration of 10 μg/mL. The coating was performed overnight then the plates were washed with PBS. NP cells were purchased and cultured using protocols from Science Cell (catalog number: 4800). Cell seeding density was 50,000/mL. Media formulation was 10% FBS, 1% NP cell growth supplements (from Science Cell; Catalog numbers 0010, 0503 and 4852), and 1% penicillin/streptomycin. Media was changed every other day.

### Example 1I. In Solution ROS Detection

H₂O₂ scavenging by MnO₂ nanomaterials was monitored by calculating the remaining H₂O₂ amount after incubation with the nanomaterials. Specifically, into 1 mL 10 mM H₂O₂ solution, 10 mg of nanomaterials were added, which is associated with bubble formation based on the catalytic conversion of H₂O₂ into oxygen and water. After 30 minutes, the mixture was centrifuged down at 10,000 rpm to remove nanomaterial residues, and the supernatant was added into a 10 mM KMnO₄ aqueous solution. After incubation in 37° C. for 1 hour, the purple color of KMnO₄ was monitored by plate reader using absorption peak at 570 nm. The amount of H₂O₂ is proportional to the intensity decrease of the KMnO₄ absorption. As a background control, PBS was added to H₂O₂ and the decrease in purple (KMnO₄) color was used for normalization. This experiment was repeated 3 times.

### Example 1J. Nanoscaffold Co-Culture with Macrophage and Condition Media Collection

Macrophages were converted from THP-1 monocytes through PMA activation. Then different scaffold conditions, including chitosan nanoscaffold, 3D-PHP nanoscaffold, and control (PBS only), as well as 3D-PHP nanoscaffold loaded with low (50 μg), medium (100 μg) and high (200 μg) dosages of BETi (JQ-1 from Tocris) drugs were added into the macrophage cultured followed by the addition of 1 μg/mL LPS for stimulation of macrophages. Four hours later, media was changed into a formulation without LPS, and macrophages continued to express pro-inflammatory proteins for 24-48 hours. Afterwards, the media was collected and centrifuged down at 13,500 rpm for 30 minutes to remove any aggregates from the nanoscaffolds. The supernatants were then collected and stored at −80° C. before mixing with disc cell media at 1:1 ratio and treatment to disc cells in the survival study.

### Example 1K. Intracellular ROS Detection

Intracellular ROS detection was performed using a standard 2′,7′ dichlorofluorescin diacetate (DCFDA) assay (ThermoFisher). Specifically, a 10 μM DCFDA solution was prepared by diluting 20 mM DCFDA DMSO solution in media (in absence of phenol red). Macrophages were converted from THP-1 monocytes using the protocol described above and cultured at a cell density of 10000 cells per well in a 24 well plate. Freshly prepared DCFDA solution was added into the adherent macrophage culture with different conditions (pre-treated with LPS in the co-culture of control and 3D-PHP nanoscaffolds). Half hour incubation at 37° C. in the incubator, the cells in 24 well plate were imaged with green fluorescence using a Nikon epifluorescent microscope. ROS levels inside cells were proportional to the fluorescent intensity of the cells. Quantification was performed by assigning a baseline fluorescent intensity (for non-H₂O₂-treated cells) then the number of green fluorescent positive cells were counted and divided to the total number of cells shown in the brightfield.

### Example 1L. Nucleic Acid Detection by YOYO-1 Assays

Nucleic acid (NA) binding by 3D-PHP nanoscaffold and control nanoscaffolds was measured by comparing the amount of NA before and after co-incubation. NA concentration was detected by YOYO-1 dye (ThermoFisher). 1 μL of 1 mM stock DMSO solution of YOYO-1 dye was diluted to 10 mL solution using TE buffer composed of 10 mM Tris, 1 mM EDTA with a pH of 8. The solution was then mixed with NA solution at a 1:1 ratio followed by incubation for a few minutes before the fluorescence intensity was measured by a plate reader. Background fluorescence was subtracted using TE buffer with YOYO-1 dye only. The percentage of NA scavenging was quantified through normalization of fluorescence intensity after NA incubation with nanoscaffolds or nanomaterials to the original NA solution. Three measurements were taken per condition.

### Example 1M. Quantitative Real Time Polymerase Chain Reaction (qRT-PCR)

Cells were lysed with Trizol®, and then the lysates were stored at −80° C. until ready for use. RNA precipitation and extraction was then performed using standard solvents and procedures suggested by the vendor. Complementary DNA sequences (cDNA) were converted from the extracted mRNA with the SuperScript™ III DNA synthesis system (Life Technologies). Power SYBR™ Green Master Mix was then added into the cDNA solution followed by qPCR on a PCR instrument (StepOne Plus from Applied Biosciences).

### Example 1N. Live Dead Assay

Effects of 3D-PHP nanoscaffold-mediated anti-inflammation on the survival or proliferation of disc cells (nucleus pulsus cells) were studied using a standard live dead assay (ThermoFisher). Specifically, live cells were stained using Calcein AM dye, which is cell-permeant only when cells were alive based on their ubiquitous esterase activities. Dead cells were stained with ethidium homomdimer (EthD)-1 dye, which only enters cells when the cell membrane become permeable. Calcein AM dye has an excitation of 490 nm and emission of 520 nm. EthD-1 dye has excitation and emission of 495 nm and 635 nm, respectively. In the detection, 2 μM Calcein AM and 4 μM EthD-1 dye were used and imaging was performed under a Nikon Ti series epifluorescent microscope. Incubation was performed at 37° C. for 30 minutes before imaging. Quantification of the live/dead cell ratio was performed by counting the number of green and red fluorescent cells within the same microscope view.

### Example 10. Nucleotomy Procedure and Implantation of Nanoscaffolds

Rats were anesthetized by injection of 2 mg/kg midazolam and 0.15 mg/kg medetomidine and butorphanol tartrate. After rats reached deep anesthesia, which was confirmed by tail pinching, posterior midline incision was performed at the Co6-7 level to expose the intervertebral disc. A no. 11 scalpel was used for the incision of posterior AF and the longitudinal cut was around 2 mm. Next, a total nucleotomy was achieved with a microcurette. After the nucleotomy, nanoscaffolds were pushed into the empty space using a micropipette tip. As controls, PBS was injected into the empty space after the nucleotomy. A heating pad was provided underneath the rats throughout the procedure and the rats were observed during recovery from the anesthesia.

### Example 1P. Von Frey Test for Pain Measurement

Pain associated mechanical allodynia was assessed by a Von Frey test. At different time points before and 2 days, 1 week, 2 weeks, 3 weeks, 4 weeks, 5 weeks, and 6 weeks after nucleotomy, rats were individually transferred into an enclosure device and accommodated to the environment for around 20 minutes in order to minimize interferences from exploratory activities. Then a 2-g filament was placed on top of the ventral surface of each tail for around 6 seconds to exert force. Only responses occurring within 6 seconds were considered. These responses included licking, flinching, shaking, and withdrawing. Negative response with value zero was assigned to rats who did not exhibit any responses. Each animal was studied with the Von Frey test for five times to obtain a consistent readout and two observers were assigned to observe the same rat without knowing which group the rat belongs to.

### Example 1Q. In Vivo MRI

MRI was performed on rats 6 WPI using a Bruker BioSpec (9.4 T) MRI scanner in order to investigate the regeneration of discs after the nucleotomy, which can be quantified by the signal intensities and water content through T2-weighted MRI. In the MRI study, time-to-repetition was set as 5,000 ms; time-toecho was set to 30 ms; and coronal axial planes were imaged for the rat discs. MRI index and signal intensities were used for the calculation of degree of damage of coccygeal disc tissues. Region of interest (ROI) was defined as the higher intensity area in the coronal plane and ROI was quantified using Fiji (Image J). MRI index was measured with blind tests by two independent observers.

### Example 1R. Histological Analysis on Anti-Inflammation and Disc Regeneration Outcome from Nanoscaffold Treatment

At 6 WPI, animals were all sacrificed and disc tissues were harvested and fixed using 10% buffered formalin for one week. Decalcification was then performed using Rapid Cal Immuno™ (BBC biochemical) for another 2 weeks. Disc tissues were then paraffin-embedded and sectioned in the coronal directions with a Leica microtome. After dewaxing, rehydration, and permeabilization by methanol, H&E, immunohistofluorescent, and Safranin-O staining were performed on different sections. For H&E staining, tissue sections were stained in a solution with Mayer's Hematoxylin for 5 minutes then washed with water and counterstained in alcoholic solution of eosin for another minute without rinsing. Tissues were then dehydrated with 80%, 90%, 95%, and 100% EtOH for one minute each followed by clearance in Xylene solution for 3 times (1 minute each) and coverslip mounting. For the Safranin-O staining, tissues were stained for 10 minutes by Weigert's iron hematoxylin solution followed by extensive washing with water. Then fast green solution was used for the second staining for 5 minutes followed by brief rinsing with 1% acetic acid solution and then stained with 0.1% Safranin-O solution. Afterwards, tissues were dehydrated and cleared using identical protocol as mentioned in the H&E staining. Safranin-O staining score was based on an 8-point scale previous used for IVD tissue analysis, and if no degenerative features were found a zero score was assigned. For the immunohistofluorescent and immunohistochemistry staining of inflammatory (e.g., MMP13 and IL1β), ECM (aggrecan, Collagen II) and pain (e.g., CGRP) markers, disc tissues with fixation and decalfication were first dewaxed, sectioned into 10 μm sections, and stained with primary antibodies followed by 24 hours incubation. After washing with PBS containing 0.1% Tween 20, the secondary antibodies were added and incubated for 2 hours. Finally, the nucleus was stained with DAPI for 15 minutes. Sections were then examined under a fluorescent or colorimetric microscope with a Zeiss 880 confocal microscope and Olympus microscope, respectively. ImageJ was used for quantification in an automatic manner.

### Example 1S. In Vivo Statistical Analysis

For the statistical analysis of data, GraphPad Prism (version 5.01, GraphPad Software) was used, and Image J software (imagej.nih.gov/ij/) was used for the quantification of data. Data are presented as mean±standard error of the mean (SEM). One-way analysis of variance (ANOVA) with the Tukey post-hoc test was used to assess effects of multiple treatments in in vivo experiments, p-values<0.05 were considered statistically significant.

### Example 1T. Statistical Analysis

OriginLab 9.1 and GraphPad Prism 5.01 were used for generating the graphs and performing the statistical analysis. Quantifications of the tissue sections were performed using ImageJ software. Data in the bar graphs represents mean±SD, N=3 biological replicates unless otherwise indicated. For multi-group analysis, One-way analysis of variance (ANOVA) and Tukey post-hoc test were used. P*<0.05, P**<0.01, and P***<0.001.

### Example 1U. Animal Experiment Protocol

The animal experiments were conducted in accordance with the guidelines approved by the Institutional Animal Care and Use Committee (IACUC) of CHA Bundang Medical Center (IACUC 200141).

### Example 2. Generation of 3D-Porous Hybrid Protein (3D-PHP) Nanoscaffolds

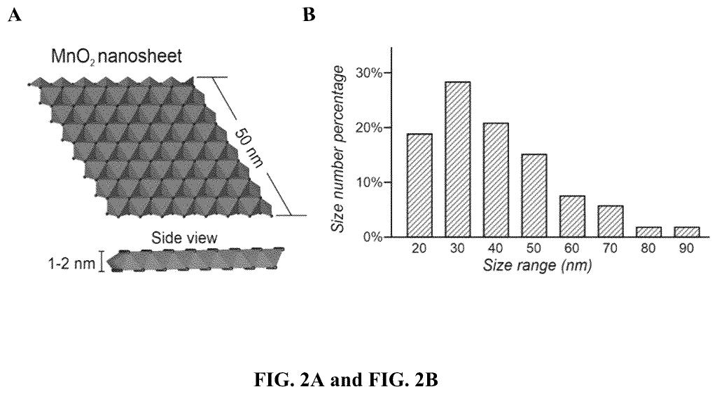

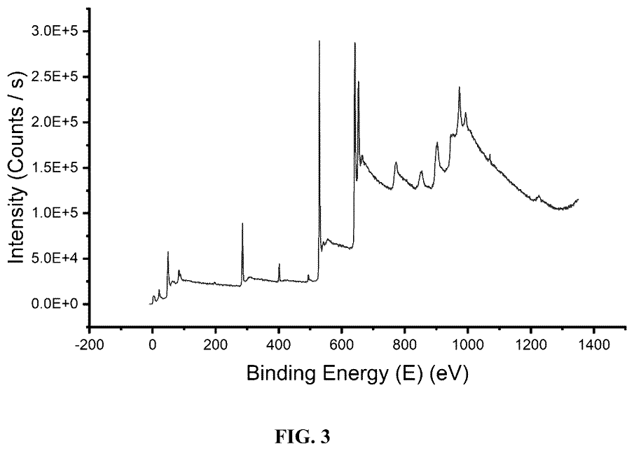

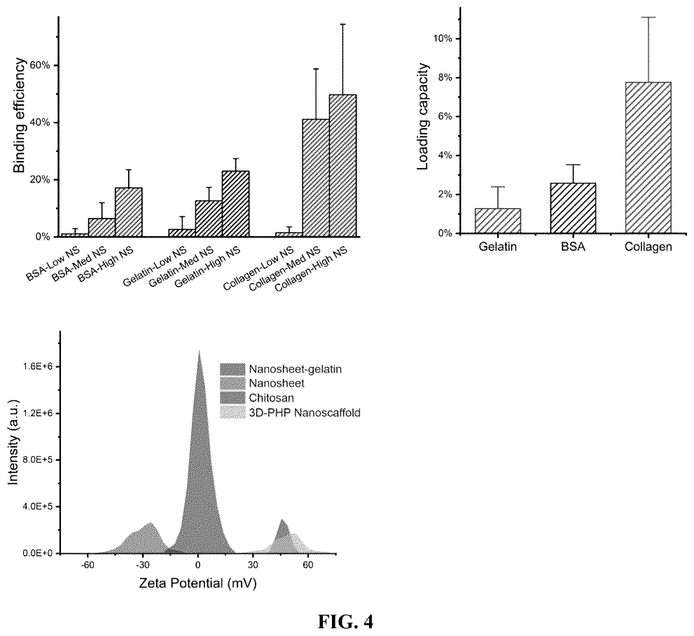

3D-PHP nanoscaffolds were created for drug delivery and tissue engineering using the nanomaterial-templated protein assembly (NTPA) approach. Protein nanoscaffolds have great promise for drug delivery and tissue engineering; yet proteins, such as gelatin, do not necessarily efficiently self-assemble under physiological conditions. The inventors speculated that nanomaterials with large surface areas and binding affinities to proteins could be utilized as a template for forming scaffolds without the need for specialized secondary structures or covalent modifications to proteins. The MnO₂ nanosheet was selected as a template due to its high binding affinities to common functional groups (—NH₂, —CONH—, —OH, —C₆H₅, etc.) in proteins through electrostatic, hydrogen bonding, and metal-π interactions, according to previous computational and experimental studies (FIGS. 2A-2B and 4). (L. Yang, et al. Nature Communications 2018, 9, 3147; G. Dey, et al. The Journal of Physical Chemistry C 2018, 122, 29017; C. Rathnam, et al. Science Advances, 7, eabj2281; and B. B. Ding, et al. Advanced Materials 2020, 32).

Compared to other 2D nanomaterials (e.g., graphene oxide) with high surface areas and protein binding affinities, the MnO₂ nanosheet is biodegradable, which is crucial for tissue engineering, and the degradation product [Mn(II)] provides magnetic resonance imaging (MRI) enhancement. Subsequently, gelatin absorption on MnO₂ nanosheets was examined by incubating the nanosheets in gelatin solution under physiological conditions (37° C., in phosphate-buffered saline (PBS)) followed by centrifugation to separate absorbed and non-absorbed gelatin. The high protein absorption was confirmed based on the peptide content in the absorbed portion of the nanosheet detected by the bicinchoninic acid (BCA) assay and the zeta potential measurements, with a shift from negative charge to almost neutral (FIG. 4). In this way, the efficient non-covalent assembly of protein onto nanomaterials was validated.

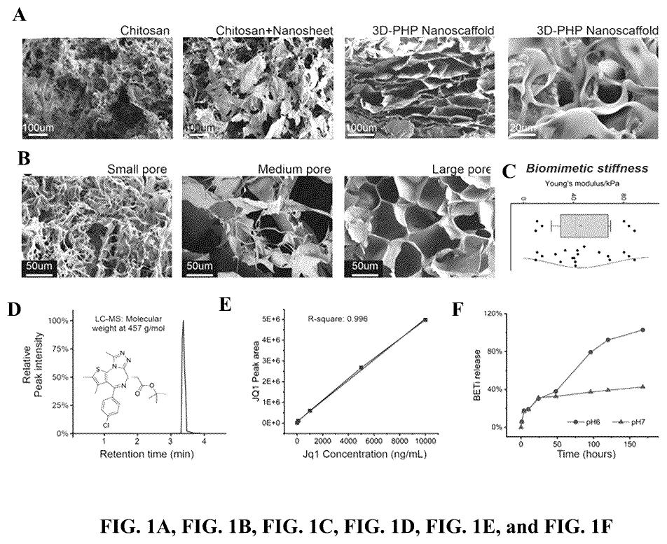

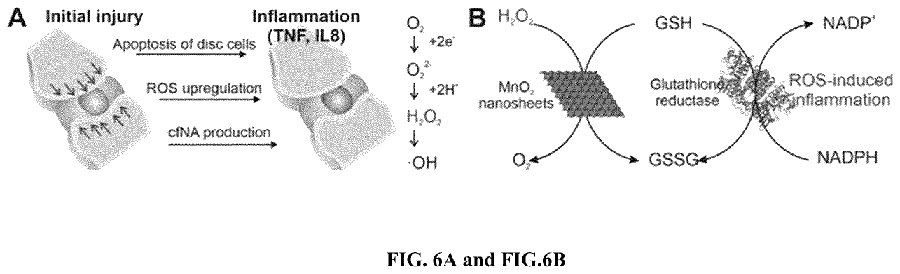

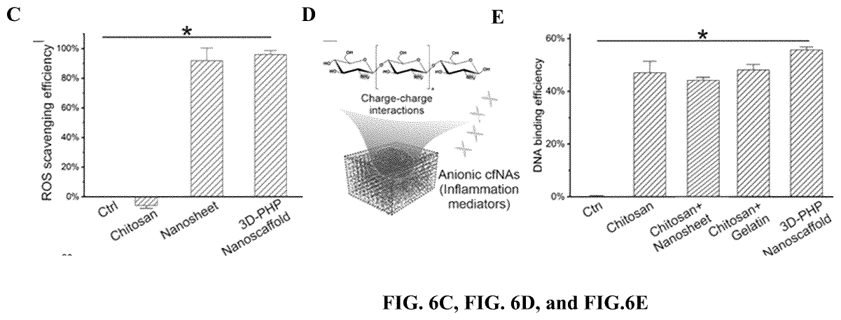

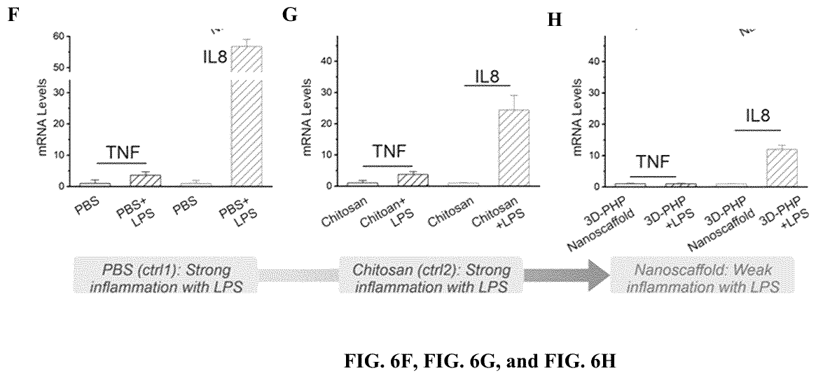

Next, an electrostatically driven interfacial assembly technique was adapted to form 3D-PHP nanoscaffolds from protein-decorated nanosheets. Specifically, a viscous droplet (1 wt %) of chitosan was incubated within aqueous solutions with varying concentrations of gelatin-bound nanosheets that initiated electrostatically driven interfacial assembly through the negative charges on the nanosheets and positive charges of chitosan. (L. Yang et al. Advanced Materials 2020, 32, 2002578 and B. Gong et al., Exploration 2022, 2, 20210035). As cationic chitosan slowly diffuses into the solution with anionic nanosheets, the water trapped between different diffusion layers of chitosan can become micropores after freeze-drying. The reverse of the negative charge to the positive charge of nanosheets in Zeta potential measurements and the porous morphology with 3D interconnected sheet-like structures in scanning electron microscopy (SEM) confirmed the synthesis of 3D-PHP nanoscaffolds (FIG. 1A). Since the size of the hydration layer during electrostatically driven interfacial assembly determines porosity, the porosity of 3D-PHP nanoscaffolds was modulated by varying the concentrations of nanosheets from 1.2, 2.4, to 3.0 mg/mL (FIG. 1B and FIGS. 6A and 6B). Thus, the mechanical properties and drug release rates of 3D-PHP nanoscaffolds could also be controlled. Given that healthy discs have stiffness in the tens of kPa, the concentration of 2.4 mg/mL nanosheet was considered optimal to form the 3D-PHP nanoscaffold, as it produces a similar stiffness range of around 30 kPa (FIG. 1C).

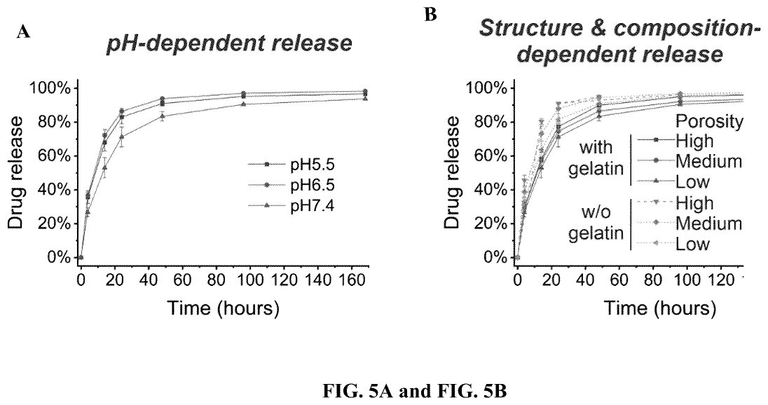

Smart biomaterial-mediated stimuli-responsive and regulated release of anti-inflammatory drugs can improve dynamic and effective inflammation modulation. In inflammatory responses associated with disc injury (e.g., nucleotomy), inflammation is highest within the first few days after injury, followed by a subacute and chronic phase of a slight decrease in inflammatory signals. Furthermore, higher levels of inflammation and apoptosis in the initial stage of injury are commonly associated with a more acidic pH in the microenvironment. Chitosan is a pH-responsive polymer, with neutral charges at physiological pH (7.4), but becoming positively charged and hydrophilic at a slightly acidic pH (below 6.5). Given that the nanoscaffold releases drugs through a diffusion-driven mechanism, it was hypothesized that the 3D-PHP nanoscaffold would release drugs more rapidly in response to stronger inflammatory stimuli. The pH-dependent drug release assay supported this hypothesis, with a significantly faster release of a model drug (rhodamine B) and the BETi (JQ1) from the 3D-PHP nanoscaffold at acidic pH (6.5) compared to neutral pH (7.4) (FIGS. 1D-1F and FIG. 5A). Besides pH, scaffold properties, including their porosities and composition, were also found to play roles in modulating drug release rate (FIG. 1E). A more porous nanoscaffold made from 1.2 mg/mL nanosheet solutions resulted in faster release rates than the experimental condition (made from 2.4 mg/mL) (FIGS. 6A and 6B). Incorporating nanosheets and gelatin into the scaffold prolonged release. In summary, a substantially faster release in the first 1-2 days, followed by a slower release by the nanoscaffold and a faster release at acidic pH, indicates that 3D-PHP nanoscaffolds have the potential for dynamic and effective control of inflammatory signals in injury-associated IVDD.

### Example 3. Dual-Scavenging of ROS and Cf-NAs by 3D-PHP Nanoscaffolds

Dysregulated inflammation can be induced by key mediators, including ROS and cf-NAs, which stimulate the pro-apoptotic and toll-like-receptor (TLR) pathways within inflammatory cells (e.g., macrophages), respectively (FIG. 6A). Nanomedicine-based approaches have been developed for scavenging ROS and cf-NAs, both of which have led to anti-inflammatory outcomes; however, scaffold-mediated dual-scavenging of both inflammatory mediators can be advantageous as it provides localized modulation of immune cells. MnO₂ nanosheets are known to scavenge ROS by catalyzing the conversion of H₂O₂ into water and oxygen, which blocks the downstream pathways of glutathione (GSH) oxidation and NADP+ production (FIG. 6B). Chitosan has also been a widely used NA carrier and thus could potentially scavenge cf-NAs. Because of the very porous structure of the 3D-PHP nanoscaffold, it was reasoned that ROS and cf-NAs might quickly diffuse through and be efficiently dual-scavenged by the nanoscaffold.

To further investigate ROS scavenging by the 3D-PHP nanoscaffold, the 3D-PHP nanoscaffold, control scaffolds (chitosan, chitosan assembled with the nanosheet), MnO₂ nanosheets were incubated with equivalent manganese content and PBS with a standard H₂O₂ solution (FIG. 6C). These reactions are often accompanied by the rapid production of oxygen bubbles when the 3D-PHP nanoscaffold and MnO₂ nanosheets are placed in the H₂O₂ solution, suggesting the well-preserved ROS scavenging capacity of the 3D-PHP nanoscaffold. In contrast, control nanoscaffolds and PBS did not show the same phenomenon. Quantifying the remaining amount of H₂O₂ using a colorimetric assay under the same experimental and control conditions confirmed this observation, as the 3D-PHP nanoscaffold and MnO₂ nanosheet conditions resulted in a reduction of 90% and 95% of H₂₀₂, while the other conditions showed minimal reduction (FIG. 6C). The ROS scavenging assay was repeated in vitro in a human macrophage model. Specifically, suspension culture of THP-1 human monocytes was converted into adherent macrophages by phorbol myristate acetate (PMA) and then treated with a 50 μM H₂O₂ solution incubated with a 3D-PHP nanoscaffold, control scaffolds (chitosan, chitosan assembled with nanosheet), and PBS. If extracellular H₂O₂ were successfully scavenged by 3D-PHP nanoscaffolds, a reduced ROS signals within cells would be observed. To confirm this, a well-established 2′,7′-dichlorofluorescein diacetate (DCFDA) kit was used to determine the intracellular ROS levels within H₂O₂-treated macrophages. The condition treated with 3D-PHP nanoscaffolds showed a significant decrease (nearly 10-fold) in ROS production compared to those treated with control scaffolds or PBS. This indicates that the 3D-PHP nanoscaffold has an excellent ROS scavenging property, as confirmed by the in-solution test. These results illustrate that the 3D-PHP nanoscaffold can serve as a therapy ROS-related diseases.

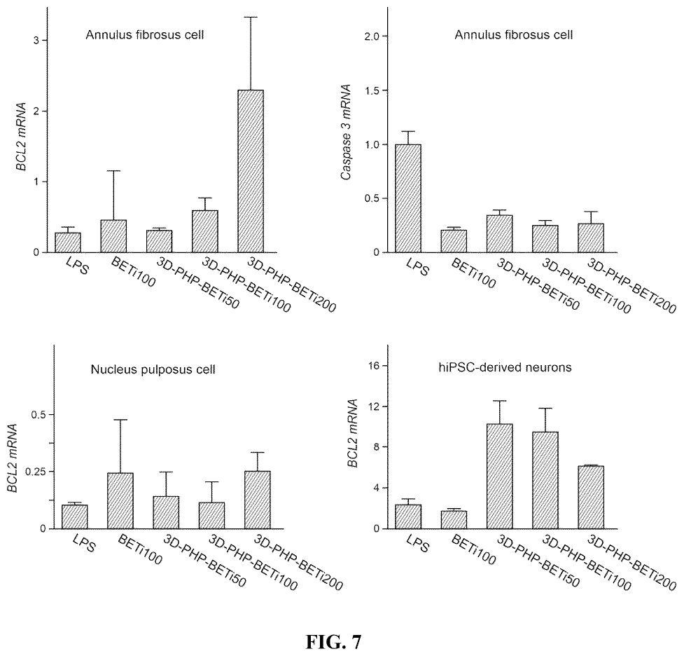

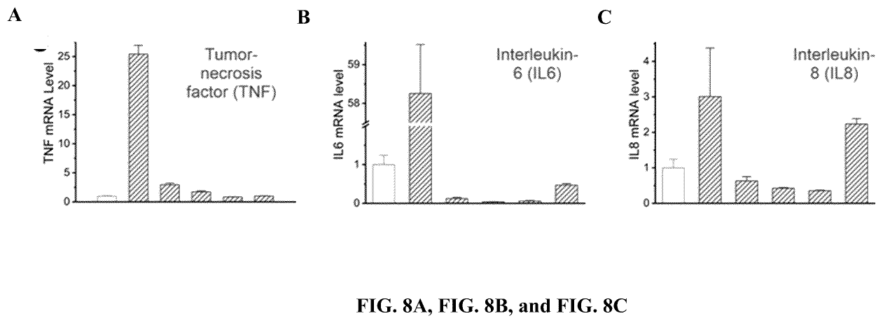

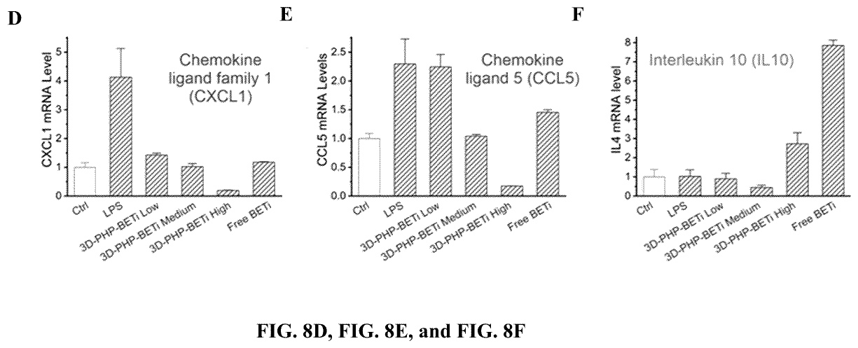

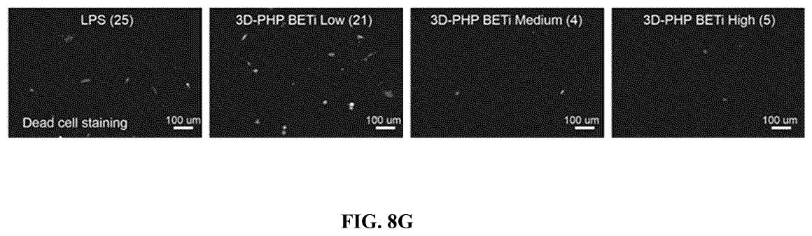

Then the cf-NAs scavenging capability of 3D-PHP nanoscaffolds was tested, comparing their NA binding efficiency with control scaffolds (chitosan and chitosan mixed with gelatin) (FIGS. 6D-6E). For this assay, Cytosine-phosphorothioate-guanine (CpG) oligodeoxynucleotides was selected as a model NA, as it has been widely associated with chronic inflammation in diseases including cancer metastasis and inflammatory bowel syndrome (IBS). YOYO™-1 Iodide dye was used to quantify the CpG amount in the solution before and after incubation with 3D-PHP nanoscaffold and control conditions. Chitosan, chitosan-gelatin, and the 3D-PHP nanoscaffold showed significant (approximately 50%) binding and scavenging of NA compared to the condition treated with PBS (FIGS. 6E and 8). The three nanoscaffolds did not show a clear difference in overall binding efficiency (FIGS. 6F and 7). This strongly indicates that chitosan plays a crucial role in CpG scavenging, and the formation of the nanoscaffolds well preserved the NA scavenging capability of chitosan polymers.

To investigate the anti-inflammatory outcome of 3D-PHP nanoscaffold-mediated dual scavenging of ROS and cf-NAs, THP-1-derived macrophages were stimulated with lipopolysaccharide (LPS), followed by incubation with 3D-PHP nanoscaffolds, control scaffolds (chitosan only), and PBS (FIGS. 6G-61). The inflammatory responses of macrophages were studied using a quantitative real-time polymerase chain reaction (qRT-PCR) based on mRNA levels of tumor necrosis factor (TNF) and interleukin 8 (IL8). From the qRT-PCR analysis, as expected, LPS treatment leads to an apparent increase in the expression of inflammatory genes for all different conditions. Chitosan alone showed slightly lower inflammation with a decrease in IL8 but not TNF. However, the 3D-PHP nanoscaffold, compared to the PBS and chitosan nanoscaffold, demonstrated the lowest expression of both TNF and IL8. Collectively, these results suggest that the scavenging of ROS and cf-NAs from 3D-PHP nanoscaffold could synergize for anti-inflammatory modulation.

### Example 4. Delivery of Epigenetic Modulators for Anti-Inflammation

Associated with inflammatory mediators (e.g., ROS and cf-NAs) there are often abnormal epigenetic modifications on the inflammatory genome of macrophages that lead to chronic inflammation after initial tissue injury. For example, BET proteins, considered as “readers” of histone acetylation, play a key role in coordinating the transcription of inflammatory genes. Treating macrophages with BETi can efficiently inhibit inflammatory cascades with excellent IC₅₀, regardless of the types of inflammatory mediators. Therefore, substantial evidence has shown the promise of BETi in treating IVDD. However, one concern about BETi for inflammatory IVDD is their potential effects on non-inflammatory biological activities, such as the suppression of cartilaginous tissue differentiation that is desired for disc repair. Thus, 3D-PHP nanoscaffolds that can provide localized, dynamic, and stimuli-responsive release of drugs could improve the outcome of BETi, while suppressing ROS and cf-NAs for combined anti-inflammation.

A Transwell® system was applied to check the anti-inflammation outcome of nanoscaffold-mediated BETi delivery. 3D-PHP nanoscaffolds loaded with 50 (3D-PHP-BETi Low), 100 (3D-PHP-BETi Medium), and 200 (3D-PHP-BETi High) μg/mL BETi were incubated with THP-1-derived macrophages stimulated with LPS, and a condition without LPS treatment was included as a control. Subsequently, macrophages were harvested for qRT-PCR analysis on pro- and anti-inflammatory genes. As expected, LPS treatment led to dramatic (from 2- to 58-fold) upregulation of all pro-inflammatory genes, including TNF, interleukins (IL 6 and IL8), chemokines (CXCL1, CCL5), and reduction of anti-inflammatory genes (IL10), and the treatment of free BETi (delivered by DMSO) significantly lowered the inflammatory responses (FIGS. 8A-8F). Most importantly, BETi-loaded 3D-PHP nanoscaffolds showed dose-dependent anti-inflammatory effects (except for the IL10 expression where the 3D-PHP-BETi low is higher than the 3D-PHP-BETi medium group (the difference is not significant at a P value of 0.05)), with an even more substantial effect than the free drug in terms of gene expression of IL6, IL8, CXCL1, and CCL5, indicating combinatorial effects from nanoscaffold-mediated scavenging of inflammatory mediators.

Furthermore, also investigated were the effects of nanoscaffold-mediated anti-inflammation on the survival and proliferation of disc cells and human induced pluripotent (hiPSC)-derived neurons, which is an essential step in disc tissue regeneration and pain reduction, respectively, after disc injuries (FIG. 8G). After treating LPS-stimulated macrophages with 3D-PHP nanoscaffolds loaded with varying concentrations of BETi, the condition media was harvested and administered to nucleus pulposus cells and neurons for 48 hours. It was hypothesized that an efficient suppression of anti-inflammation in macrophages could reduce cytotoxic cytokines such as TNF, reducing stress and apoptosis in both cell types. Through the use of a live/dead assay, it was shown that 3D-PHP and BETi-loaded nanoscaffold offered more effective protection for cells than BETi-treated or nanoscaffold-treated conditions. Thus, the 3D-PHP nanoscaffold can modulate dysregulated inflammatory signals due to its anti-inflammatory effects and combination with the delivery of epigenetic modulators.

### Example 5. Enhanced Treatment of IVDD by 3D-PHP Nanoscaffolds

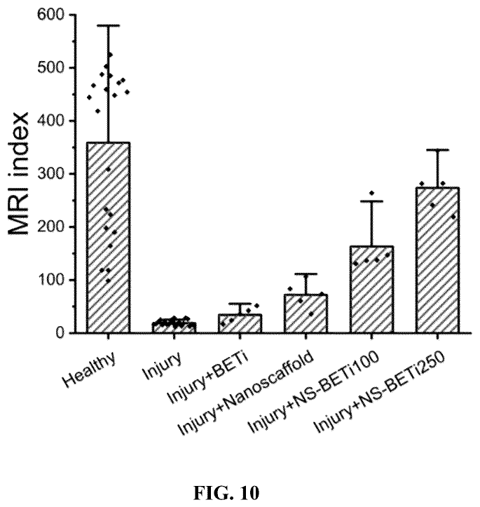

With promising in vitro results, the in vivo anti-inflammatory outcome of BETi-loaded 3D-PHP nanoscaffolds in the context of IVDD was further evaluated. Because it has a high level of reproducibility and is relatively noninvasive, the rat nucleotomy model was selected as the best way to induce disc degeneration. It also allows the direct comparison of injured and healthy discs within the same animal. (G. Vadalà et al., Tissue Engineering Part C: Methods 2015, 21, 1117). Immediately after nucleotomy, massive apoptosis of disc cells with ROS and cf-Nas secretion will occur within the nucleus pulposus (NP) and annulus fibrosus (AF) regions that initiate an acute phase of intense inflammation followed by subacute and chronic phases of inflammation in the next few weeks. Thus, 3D-PHP nanoscaffolds loaded with medium (assembled with 100 μg/mL) and high (assembled with 200 μg/mL) BETi (denoted as 3D-PHP-BETi Medium and 3D-PHP-BETi High, respectively) were implanted into the disc within the same day of injury, and their performance was compared in terms of inflammation and tissue regeneration. 3D-PHP nanoscaffolds, free BETi, and PBS conditions were also implanted into nucleotomized rat intervertebral discs as controls. At six weeks-post injury (WPI), animals were sacrificed, then histological analysis of disc integrity based on quantifications of hematoxylin and eosin (H&E) staining and MRI index (FIG. 10). Compared to rats without nucleotomy, the nucleotomy model was validated based on the massive decrease (over 95%) of 6-WPI healthy disc tissue. Although the injection of BETi slightly improved the restoration of the disc structure, the enhancement is minimal compared to the 3D-PHP-BETi Medium and 3D-PHP-BETi High groups. Interestingly, the 3D-PHP nanoscaffold itself also promoted the preservation or regeneration of disc tissues, which is consistent with the in vitro data on nanoscaffold-based dual-scavenging of ROS and cf-NAs. As a result, it was hypothesized that the anti-inflammatory effect of the 3D-PHP nanoscaffold and the regulated distribution of BETi were the factors responsible for the increased regeneration of disc tissue.

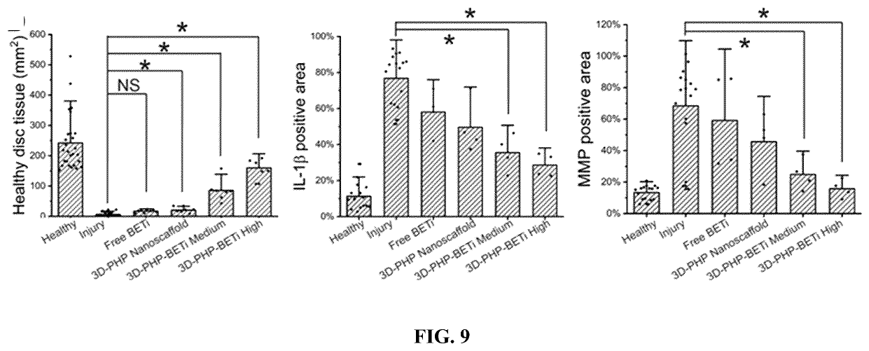

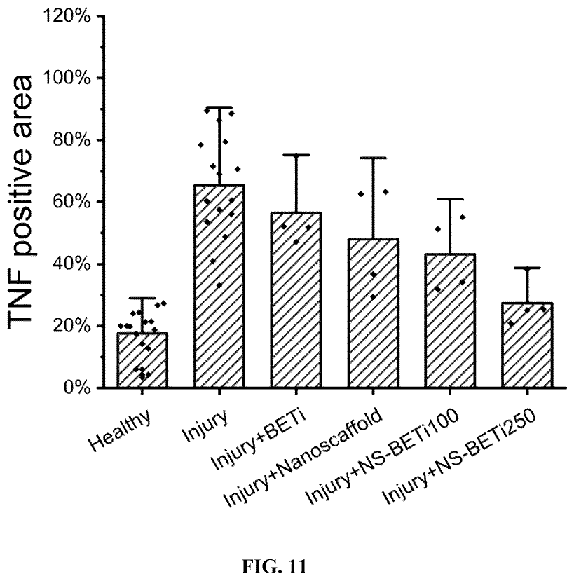

IL-1β and TNF immunostaining was also performed to verify the hypothesis on the injured disc tissues (FIGS. 9 and 11). IL-1β is a well-established chronic inflammatory marker that is often associated with pain. Based on quantifying IL-1β positive areas 6WPI in animals treated with experimental and control conditions, similar trends were observed as shown in H&E staining, directly supporting the hypothesis on the correlation between anti-inflammation and tissue regeneration. Specifically, nucleotomy significantly upregulated IL-1β by more than 7-fold, consistent with previous reports, and 3D-PHP-BETi High significantly suppressed IL-1β expression 6WPI. On the contrary, BETi injection alone did not induce a significant reduction of IL-1β, which could be attributed to the rapid diffusion of free drugs after injection into the disc. Analysis of another inflammatory marker, matrix metalloproteinase-13 (MMP-13), which is often associated with the degradation of healthy disc ECM during its degeneration, showed a consistent trend, with almost complete restoration of MMP-13 levels in healthy tissues after treatment with 3D-PHP-BETi High nanoscaffold. At the same time, BETi injection alone did not suppress the MMP-13 levels compared to injury-only conditions (FIG. 9). These results supported the strong correlation between anti-inflammation and tissue regeneration.

### Example 6. ECM Regeneration and Pain Reduction by Anti-Inflammation

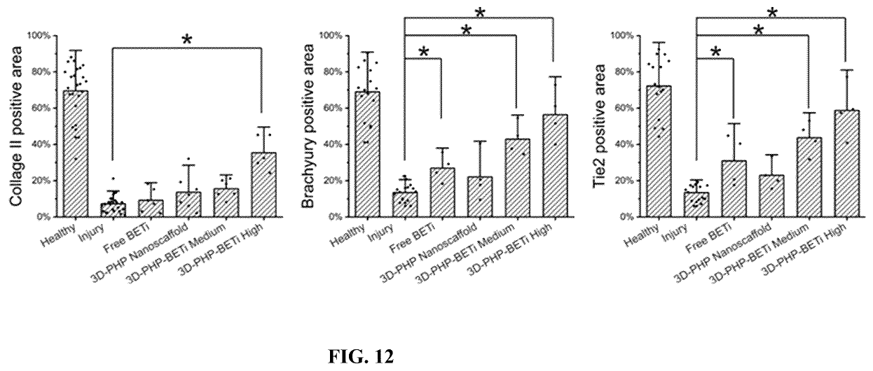

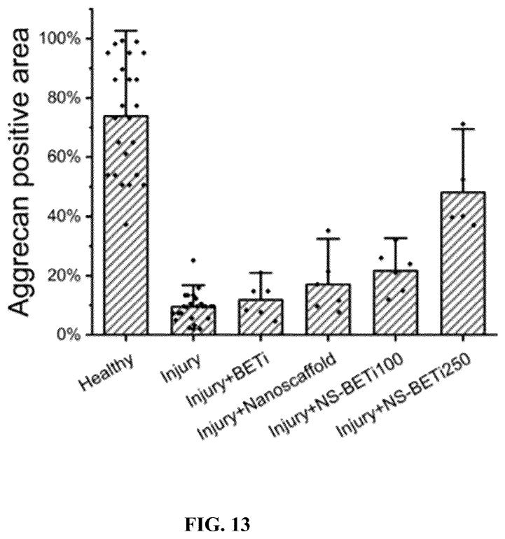

Another aim was to understand the mechanism of disc tissue regeneration. Significant MMP-13 reduction was observed in the 3D-PHP-BETi High nanoscaffold condition that showed the strongest tissue regeneration in H&E staining; recent evidence has also suggested the critical role of immune cells in ECM production. Consequently, a substantial relationship was predicted between the nanoscaffold-mediated decrease of inflammation and ECM regeneration. To verify this, Collagen II and aggrecan expression 6WPI in the same cohort of nanoscaffold implantation studies was analyzed (FIG. 13). The immunohistological analysis revealed that there was an over 3-fold and 4-fold increase in the expression of Collagen II and aggrecan (which are indicative biomarkers of a healthy disc ECM in the nucleus pulposus), respectively, in the 3D-PHP-BETi High condition. At the same time, free BETi did not result in noticeable up-regulation. Although not statistically significant, the 3D-PHP nanoscaffold alone resulted in a 30% increase in Collagen II compared to the injury-only group, which could be attributed to anti-inflammatory effects or gelatin-facilitated ECM regeneration, according to the literature. In addition to the up-regulation of healthy disc ECM components, an increase in disc cell populations was observed in the same area of disc tissues with 3D-PHP-BETi High and 3D-PHP Medium treatment, based on quantification of Brachyura and Tie2 positive areas in tissue immunostaining (FIG. 12). The strong correlation of the increase in disc cell populates in the same area of disc tissues with 3D-PHP-BETi High and 3D-PHP medium treatment indicated the vital role of anti-inflammation in ECM remodeling and tissue regeneration.

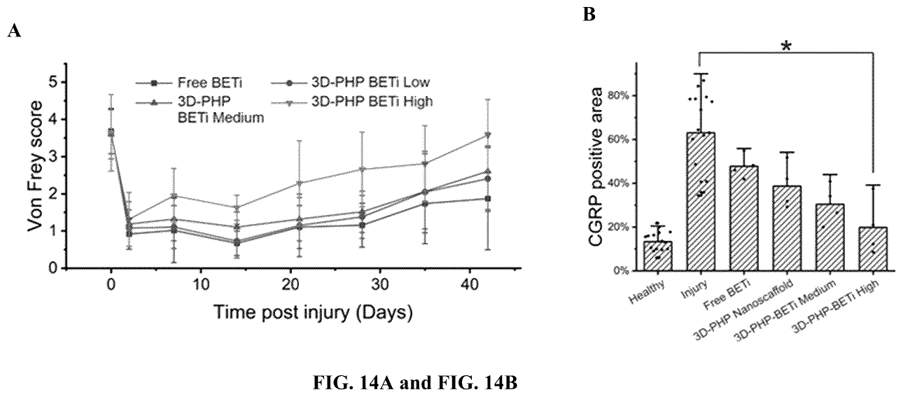

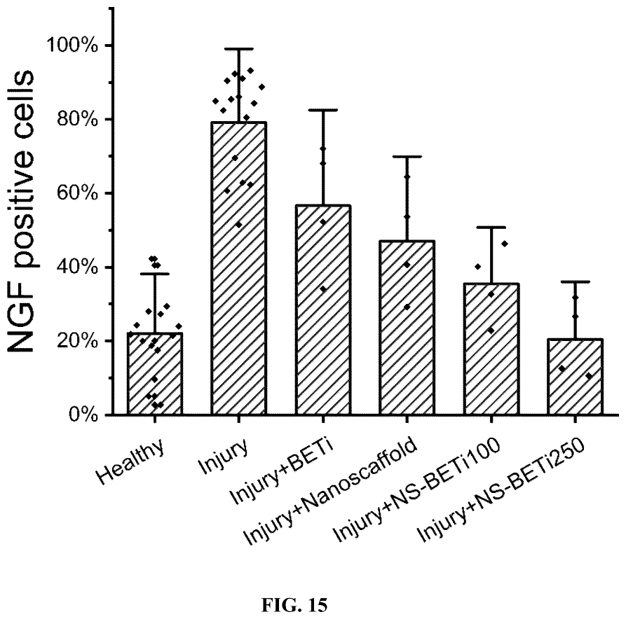

In addition to tissue regeneration, advanced therapeutic approaches that facilitate pain management after disc injuries represent another critical challenge to address. Robust anti-inflammatory treatment has been associated with pain reduction; it was also investigated whether reduced inflammation and improved disc regeneration by 3D-PHP nanoscaffolds could further reduce pain signals. The most representative pain signaling marker is a calcitonin gene-related peptide (CGRP), directly related to the alternative processing of calcitonin genes. It was determined if CGRP is activated in the rat tail nucleotomy model. In fact, a nearly 4-fold increase in the positive area ratio of CGRP was observed in rat discs after nucleotomy compared to healthy conditions (no injury) conditions. Surprisingly, 6WPI following 3D-PHP-BETi Medium and 3D-PHP-BETi High therapy significantly reduced CRGP signals compared to injury alone, whereas nanoscaffold or BETi injection alone showed considerably less reduction. Consistent with this result, Von-Frey further proved the most robust pain reduction from 3D-PHP-BETi High, compared to the BETi, nanoscaffold alone, and 3D-PHP-BETi Medium conditions (FIGS. 14A-14B and 15). The ability to reduce pain after disc injuries represents another advantage of 3D-PHP nanoscaffolds, in addition to reducing inflammation, improving tissue regeneration, and remodeling the ECM.

### CONCLUSIONS

Provided is a novel therapeutic strategy based on a nanoscaffold composed of a 3D porous hybrid protein (3D-PHP) for the treatment of IVDD. This multi-functional and multi-dimensional nanoscaffold can: i) remove inflammatory cfNAs and ROS, ii) deliver anti-inflammatory epigenetic modulators in a controlled way, and iii) promote ECM for IVD regeneration. Protein bioscaffolds are often preferred for drug delivery and tissue engineering because they are biocompatible, biodegradable, programmable, and self-therapeutic. However, the challenge with using proteins to create 3D scaffolds is that this only works for a subset of proteins with specific secondary structures (such as amyloid beta sheets), or by modifying proteins with cross-linkable ligands, which can compromise their biological properties. Modifying the drug release profile in IVDD treatments also requires modulating the porosity of protein scaffolds without altering the chemical structures or protein concentrations. To address these problems, a nanomaterial-templated protein assembly (NTPA) strategy was adopted. In this method, proteins were densely adsorbed to biodegradable 2D nanomaterials (MnO₂ nanosheets), which were then assembled with cationic polymers (such as chitosan) into 3D nanoscaffolds using an electrostatically driven interfacial assembly technique.

Gelatin (denatured collagen proteins) that does not self-assemble under physiological conditions was successfully used to form protein 3D nanoscaffolds with tunable porosities without requiring covalent modifications on the proteins. Furthermore, the addition of MnO₂ nanosheets and cationic polymers, which accelerate ROS removal and bind to NAs, allows for the self-therapeutic use of 3D-PHP nanoscaffolds. The sustainable delivery of bromodomain extraterminal inhibitor (BETi) using 3D-PHP nanoscaffolds was demonstrated for the treatment of IVDD. BETi is a highly potent epigenetic inhibitor of inflammation. The hypothesis was tested that the implantation of BETi-loaded self-therapeutic 3D-PHP nanoscaffolds in a nucleotomy IVDD rat model would effectively suppress chronic inflammation, restore healthy components of the ECM and disc cells, improve tissue regeneration, and reduce IVDD-associated pain.

The above-described results provide an innovative strategy for developing effective therapies for chronic inflammatory diseases, such as IVDD, using a biomaterial-based approach for targeted drug delivery and tissue regeneration. In addition to providing a biomimetic environment for cell growth and tissue regeneration, the 3D-PHP nanoscaffold system can enable sustained release of anti-inflammatory agents in a spatio-temporal manner. Additionally, the incorporation of self-therapeutic components into the nanoscaffolds enhances the scavenging of ROS and cf-NAs, which are known to contribute to inflammation and tissue damage. Importantly, this approach has applications not only for treating IVDD, but also for treating other chronic inflammatory conditions, such as osteoarthritis, rheumatoid arthritis, and even cancer. Besides its therapeutic applications, this approach also offers an opportunity to investigate the mechanisms underlying chronic inflammation and tissue damage. This provides valuable insights into the pathogenesis of IVDD and other chronic inflammatory diseases, which could lead to the development of more effective treatments. Furthermore, the 3D-PHP nanoscaffold system has applications in regenerative medicine, and its combination with anti-inflammatory agents could promote the repair and regeneration of multiple tissues. Notably, the nanoscaffold can be personalized to meet the unique needs of individual patients, offering customized treatment options. Consequently, the development of the 3D-PHP nanoscaffolds represents a promising platform technology for tissue engineering and drug delivery.

It will be appreciated by those skilled in the art that changes could be made to the embodiments described above without departing from the broad inventive concept thereof. It is understood, therefore, that this invention is not limited to the particular embodiments or examples disclosed, but it is intended to cover modifications that are within the spirit and scope of the present invention as defined by the appended claims.

What is claimed is:

1. A 3D porous hybrid protein (3D-PHP) nanoscaffold comprising biodegradable manganese dioxide (MnO₂) nanosheets assembled with aqueous cationic polymer solutions and extracellular matrix proteins, wherein the nanoscaffold comprises at least 100 layers, wherein the size of the cationic polymers is greater than 100 kDa, wherein the concentration of the cationic polymers is greater than 1% (weight percent to water), and wherein the nanoscaffold further comprises a therapeutic agent.

2. The nanoscaffold of claim 1, wherein the cationic polymers comprise chitosan, polyphenylalanine, polytryptophan, polyasparagine, polyglutamine, polylysine, polyarginine, polyhistidine, polyethylenimine, or poly (amidoamine).

3. The nanoscaffold of claim 1, wherein the nanosheets are assembled into the 3D-PHP nanoscaffold via an electrostatically driven layer-by-layer (LBL) 3D assembly technique and wherein the ratio of MnO₂ to proteins is about 9:1.

4. The nanoscaffold of claim 1, wherein the cationic polymer solution concentration is about 20% to about 70%, the concentration of MnO₂ is about 20% to about 70%, and the concentration of the proteins is about 5% to about 15%.

5. The method of claim 1, wherein the extracellular matrix proteins comprise, collagen I, collagen II, laminin, and fibronectin.

6. The method of claim 1, wherein the viscosity of the aqueous cationic polymer solution is greater than 20 cP (centipoise).

7. The nanoscaffold of claim 1, wherein the pore sizes range from about 5 μm to about 50 μm.

8. The nanoscaffold of claim 1, wherein the nanoscaffold further comprises a bromodomain extraterminal inhibitor (BETi).

9. A method of treating intervertebral disc (IVD) degeneration in a subject in need thereof, the method comprising administering to said subject a 3D porous hybrid protein (3D-PHP) nanoscaffold comprising biodegradable manganese dioxide (MnO₂) nanosheets assembled with aqueous cationic polymer solutions and extracellular matrix proteins, wherein the nanoscaffold comprises at least 100 layers, wherein the size of the cationic polymers is greater than 100 kDa, wherein the concentration of the cationic polymers is greater than 1% (weight percent to water), and wherein the nanoscaffold further comprises a therapeutic agent.

10. The method of claim 9, wherein the cationic polymers comprise chitosan, polyphenylalanine, polytryptophan, polyasparagine, polyglutamine, polylysine, polyarginine, polyhistidine, polyethylenimine, or poly (amidoamine).

11. The method of claim 9, wherein the nanosheets are assembled into the 3D-PHP nanoscaffold via an electrostatically driven layer-by-layer (LBL) 3D assembly technique and wherein the ratio of MnO₂ to proteins is about 9:1.

12. The method of claim 9, wherein the cationic polymer concentration is about 20% to about 70%, the concentration of MnO₂ is about 20% to about 70%, and the concentration of the proteins is about 5% to about 15%.

13. The method of claim 9, wherein the extracellular matrix proteins comprise, collagen I, collagen II, laminin, and fibronectin.

14. The method of claim 9, wherein the viscosity of the aqueous cationic polymer solution is greater than 20 cP (centipoise).

15. The method of claim 9, wherein the pore sizes range from 5 μm to about 50 μm.

16. The method of claim 9, wherein the nanoscaffold further comprises a bromodomain extraterminal inhibitor (BETi).

17. The method of claim 9, wherein the nanoscaffold delivers the therapeutic agent to the subject in need thereof.

18. A method of treating a chronic inflammation-related disease or condition in a subject in need thereof, the method comprising administering to a subject in need thereof a 3D porous hybrid protein (3D-PHP) nanoscaffold comprising biodegradable manganese dioxide (MnO₂) nanosheets assembled with cationic polymers and extracellular matrix proteins, wherein the nanoscaffold comprises at least 100 layers, wherein the size of the cationic polymers is greater than 100 kDa, and wherein the concentration of the cationic polymers is greater than 1% (weight percent to water).

19. The method of claim 18, wherein the cationic polymers comprise chitosan, polyphenylalanine, polytryptophan, polyasparagine, polyglutamine, polylysine, polyarginine, polyhistidine, polyethylenimine, or poly (amidoamine).

20. The method of claim 18, wherein the nanosheets are assembled into the 3D-PHP nanoscaffold via an electrostatically driven layer-by-layer (LBL) 3D assembly technique and wherein the ratio of MnO₂ to proteins is about 9:1.
